(19) United States
(12) Patent Application Publication
(10) Pub. No.: US 2011/0092560 A1
(43) Pub. Date: Apr. 21, 2011

(54) TOPICAL COMPOSITION FOR CONTROLLING ECTOPARASITES IN DOGS AND CATS

(75) Inventor: Mauricio Del Bigio, Sao Paulo (BR)

(73) Assignee: CEVA SANTE ANIMALE SA, Libourne (FR)

(21) Appl. No.: 12/999,682

(22) PCT Filed: Jun. 16, 2009
(86) PCT No.: PCT/BR09/00174

§ 371 Date: Dec. 17, 2010

(30) Foreign Application Priority Data
Jun. 17, 2008 (BR) PI0802255-0

(51) Int. Cl. A61K 31/415; A61P 33/14

(52) U.S. Cl. 514/407

(57) ABSTRACT

This invention describes a new topical formulation (spot-on) for controlling ectoparasites in cats and dogs, which comprises the active ingredient Fipronil and/or the derivates and salts thereof, as well as antioxidants and a suitable vehicle. The use of an organic solvent acting as the transdermal carrier provides this product with a longer residual period and, consequently, higher efficacy.

### SCOPE

This invention relates to a topical formulation (spot-on) for veterinary use in the treatment and control of parasitical diseases in dogs and cats, particularly flea and tick infestations.

### PRIOR ART

Dermatopathies, or parasitical diseases, are described as being responsible for most dermatological alterations typically found in small animals, are widely known to affect several species of domestic animals, not only due to frequency of occurrence, but also due to the medical-veterinary importance inherent to some of same.

Veterinary medicine has made important advancements in terms of knowledge, treatment and control of parasitical diseases in pets (particularly cats and dogs). The control and treatment of flea, tick, and mite infestations has been one of the primary objectives of veterinary pharmaceutical companies, not only because of the discomfort said infestations cause, but also because of the diseases that said infestations transmit to animals (babesiosis, ehrlichiosis, leishmaniosis) and to human beings (Rocky Mountains spotted fever).

Flea infestations commonly occur in domestic animals and household environments. The primary species of flea that infests dogs and cats is Ctenocephalides felis felis, infestations of which are frequent, particularly in tropical and temperate countries. Fleas are intermediate hosts of the cestode of cats and dogs, Dipylidium caninum, the dog parasitical filarial, Dipetalonema reconditum, vector of feline rickettsiosis (Rickettsia felis), cat scratch disease (Bartonella henseale), canine mycoplasmosis (Mycoplasma haemocanis), and feline mycoplasmosis (Mycoplasma haemofelis). Fleas have recently been described as being involved in the transmission of feline leukaemia and their possible participation in the epidemiology of canine leishmaniosis.

Fleas are also implicated in Flea Allergy Dermatitis (FAD) in dogs and cats, which is caused by the action of saliva, which contains allergenic substances that cause intense skin reactions in hypersensitive animals. Typical symptoms include erythema (redness), papules (bumps), pustules (pus-filled bumps), crusts (scabs) also if severe, hair loss and eczematous skin rash. These symptoms will occur often in upper tail, neck and down the back of the legs. There is no gender or age predisposition, but most cases occur in animals between two and five years of age.

Ixodidiosis, or tick infestation, which occurs in tropical countries, is caused mainly by Rhipicephalus sanguineus. This species has taken advantage of the growth of large cities and the spread of central heating installed in buildings to disseminate in urban zones, where said species frequently gives rise to enormous populations that are difficult to treat and control, causing disease to animals due to blood spoliation, which may cause rashes, itching, loss of appetite, anaemia and, in severe cases, the death of the animal. Ticks can transmit innumerable diseases to dogs, such as canine babesiosis, caused by Babesia canis, and canine ehrlichiosis, caused by Ehrlichia canis.

The acarine, Otodectes cynotis, commonly known as the ear mite, infests the ear canal of several animal species, particularly dogs and cats, causing inflammatory symptoms. Transmission occurs by direct contact, and acarines are highly contagious. The entire life cycle thereof takes place on the hosts and lasts for approximately three weeks. The ear mite is a very active parasite, and the presence thereof is usually associated with itching and increased production of secretion, which can lead to secondary bacterial and fungal infections, causing strong discomfort to animals.

Scabies, caused by a tiny and usually not directly visible parasite—the mite, Sarcoptes scabiei—which burrows under the host's skin, causes intense allergic itching. Scabies is of great veterinary importance. This disease presents endemic characteristics among humans as well as domestic and wild animals all around the world. This parasite spends the whole life cycle thereof on the host, said life cycle lasting for approximately three weeks.

Today, there are many chemical compounds that can be used to control ectoparasites in animals. Among these chemicals, the following may be cited: organochlorates (the use of which is currently forbidden), organophosphorates, pyrethrins, pyrethroids, phenylpyrazoles, macrocyclic lactones, neonicotinoids, and Insect Growth Regulators (IGR).

Fipronil is a synthetic molecule of the chemical group phenylpyrazole, the primary characteristics of which are insecticide and acaricide efficacy, a wide safety margin and strong residual power. Fipronil is indicated in the treatment and prevention of ectoparasitoses (fleas, ticks and mites) in dogs and cats. Trials have demonstrated its efficiency as an acaricide in dogs and cats by weekly topical (spray and pour-on) applications for up to 4 to 6 weeks.

Fipronil has an action mode that is different from the classic insecticides/acaricides. Fipronil is an extremely active molecule, causing the interruption of the normal function of neurons. Fipronil links to the GABA receptors, blocking the chlorine channels of neurons in the central nervous system. The GABA receptor is responsible for inhibiting neuronal activity (preventing the excessive stimulation of neurons). When the functions of the nervous system are blocked by fipronil, the result is neuronal hyperexcitement and death of parasites. Fipronil kills ectoparasites through contact with hair.

In spot on formulations, fipronil translocates by passive diffusion from the application site via the sebaceous secretions present on the hair and the skin. Regardless of the formulation, this particularity of fipronil ensures the persistence thereof in high concentrations in the hair coat of dogs and cats, ensuring the efficacy thereof even when the animals are wet or after washing. When topically applied, fipronil spreads rapidly through the epidermis and pilosebaceous units, accumulating in the sebaceous glands and being gradually released by the follicular ducts. Studies conducted on mice to assess the absorption, distribution, metabolism, excretion, and pharmacokinetics of fipronil have proven that, after oral administration, metabolites are eliminated in the faeces (45-75%) and urine (5-25%). Residues of the product were found in fat, the adrenal glands, pancreas, skin, liver, kidney, and muscle. The pharmacokinetic study demonstrated that the plasma half-life of fipronil varies between 149 and 200 hours after oral administration.

A study conducted on Beagle dogs was carried out to determine the distribution and absorption of fipronil when administered topically. In this study, fipronil was marked with ¹⁴C and administered topically in a dose of 12 mg/kg. Skin biopsies from the lumbar region were taken (5 mm²) on days 3, 7, 15, 21, 29 and 56 after application. Analysis of these biopsies determined that ¹⁴C-fipronil had high concentrations in the corneous stratus and sebaceous glands on days 7 and 56. The phenomenon of accumulation of ¹⁴C-fipronil in the sebum was found to occur by migration through the skin and hair. However, no radioactivity was detected in the hypodermis, the adipose tissue or the cells of the basal layer of the epidermis, which demonstrates that fipronil is practically not absorbed. The strong persistence of radioactivity in the skin structures and hair maintains good consistency with the duration of fipronil activity after topical application.

Fipronil has a wide safety margin because of the structural difference of the GABA receptor between invertebrates and vertebrates, justifying the safety and use thereof in female dogs and cats that are pregnant or lactating, puppies and kittens, adult and elderly animals. Studies conducted on laboratory animals proved that fipronil is not carcinogenic, teratogenic or mutagenic, demonstrating the safety of use thereof in pregnant females and young animals.

Many documents describe formulations based on fipronil and the associations thereof for controlling ectoparasites in dogs and cats. One example is the Brazilian patent application BRP19510073 3, which describes a formulation to kill fleas and ticks, the vehicles of which are a crystallisation inhibitor (polyvynilpyrrolidone) and an organic solvent (acetone). Another example would be a formulation based on fipronil associated with a growth inhibitor to control fleas and ticks (BRP19702150 4).

### OBJECTIVES

This invention describes a new formulation to be used in the control and treatment of parasitical diseases in dogs and cats, particularly flea and tick infestations. The efficiency thereof is due to the lethal action of the molecule (knock-down effect) on the ectoparasites, giving quick relief to animals. The use of an organic solvent acting as the transdermal carrier provides this product with a longer residual period and, consequently, higher efficacy.

### SUMMARIZED DESCRIPTION OF THE INVENTION

This invention relates to a topical composition for controlling ectoparasites in dogs and cats, comprising the active ingredient fipronil and/or the derivates and salts thereof, formulated with one or more transdermal carriers that facilitate the topical absorption of the product, thereby increasing the efficacy thereof in fighting against ectoparasites in cats and dogs, in addition to antioxidants and a suitable vehicle.

### DETAILED DESCRIPTION

This invention relates to a topical formulation, preferably “spot-on”, to control ectoparasites in cats and dogs, which comprises the active ingredient Fipronil and/or the derivates and salts thereof, a transdermal carrier, in addition to antioxidants and a suitable vehicle.

The “spot on” or “drop spot” composition refers to a composition of topical use that is applied to only one spot of the body of the animal (neck). From this spot, the ingredient spreads rapidly over the entire body surface, thereby providing generalized protection. Fipronil translocates through the epidermis, accumulates in sebaceous glands, and is gradually released by the follicular ducts.

The product is an innovative formulation whose composition is different because of the use of a transdermal carrier, that is, an organic solvent that facilitates the topical absorption of the product, thereby increasing the efficacy thereof in the control and treatment of parasitical diseases, particularly tick and flea infestations that affect cats and dogs.

The recommended dose of Fipronil to kill and control ectoparasites in cats and dogs is 6.7 mg/kg, with a concentration of 10% in the final product, that is, 100 mg/mL, equivalent to 0.67 mL/10 kg of the animal live weight. The formulation is mentioned in ideal proportions, and the main compound may vary according to the following range:

Fipronil at 100% . . . 80.0 to 120.0 mg/mL (80 to 120%)

The organic solvents used in the composition acting as transdermal carriers may be selected from among dimethylsulfoxide, ethylic alcohol, lactic acids, aliphatic alcohol containing 1 to 5 carbons, organic acids, propylenoglycol and the derivates thereof, isoparaffins, alkyl benzilic esters, dialkyl esters, benzil-benzylic esters, aliphatic ketones, aliphatic hydrocarbons, ethylene glycol and the derivates thereof, polyalcohol pyrrolidones and the derivates thereof, ethyl oleate.

For the purposes of this invention, the preferable transdermal carrier is dimethylsulfoxide, which can be used in concentrations of between 50% to 90% of the formulation, since data in the literature and efficacy tests carried out in the field with the target species (cats and dogs) indicate that the preferable concentration of this carrier is 80% of the formulation, that is, 80 mL of dimethylsulfoxide in 100 mL of the product.

This invention also comprises, in the formulation thereof, the inclusion of antioxidants like butylhydroxyanisol (BHA), butylhydroxytoluene (BHT), ascorbic acid, acorbil palmitate, monothioglycerol, propylgallate, sulphur dioxide, tocopherol, tocopherol acetate, oil tocopherol solutions that have the function of preserving the physical-chemical characteristics of the product. The preferable antioxidants to be used are butylhydroxyanisol (BHA) and butylhydroxytoluene (BHT).

As for the vehicle, the formulation may contain isopropyl alcohol, ethyl alcohol, or propylenoglycol, but preferably isopropyl alcohol.

The following qualitative-quantitative ranges and their respective basic components are for preparing 100 litres of solution:

### Fipronil at 100% 5.00 to 20.00 Kg

### Butylhydroxyanisol (BHA) 0.015 to 0.022 Kg

### Butyllhydroxytoluene (BHT) 0.007 to 0.011 Kg

### Dimethylsulfoxide 50.00 to 90.00 L

Isopropyl alcohol q.s.p. 100.00 L

The preferable concentrations of butylhydroxyanisol and butylhydroxytoluene are 0.18 mg/mL and 0.09 mg/mL, respectively.

According to this invention, the formulation is for controlling ectoparasites in dogs and cats, which may be fleas (Ctenocephalides felis felis and Ctenocephalides canis), mites (Sarcoptes scabiei var. canis., Notoedres cati, Otodectes cynotis) and ticks (Rhipicephalus sanguineus, Amblyoma spp., Ixodes spp.).

This formulation is also for use as an auxiliary formulation in the control of cestode (Dipylidium caninum) infestations that affect dogs and cats, as the fleas are intermediate hosts of said tapeworm. Furthermore, said formulation combats the brown dog tick (Rhipicephalus sanguineus). This is an essential fact, as this tick is responsible for the transmission of canine babesiosis and canine ehrlichiosis, diseases caused by Babesia canis and Ehrlichia canis, respectively. Said formulation also assists in the treatment and control of Flea Allergy Dermatitis (FAD) and may be used to control the mites that cause ear mange (Otodectes cynotis) and itch mites (Sarcoptes scabiei).

Below are examples that illustrate the productive process, the safety margin and the efficacy of the product for the sole purpose of better characterising the invention, but without any limitation to the formulation described herein.

### Example 1

### Process to Obtain the Product

A) In a stainless steel tank with appropriate capacity, add 90% of the dimethylsulfoxide while stirring;

B) While stirring, add the Fipronil and stir until completely dissolved.

C) Add the isopropanol, butylhydroxyanisol, and butylhydroxytoluene and stir until a clear solution is obtained.

D) Complete the volume with the remaining dimethylsulphoxide.

E) Stir for 15 minutes.

F) Filter the product into a duly cleaned and identified container using:
Pre-filter: 5-micra filtering element.
Terminal filter: 1-micra filtering element.
Stainless steel or plastic shell.

G) Collect a 100 ml sample of the product and send it for a physical-chemical analysis by Quality Control.

H) After approval by Quality Control, the product awaits the approval of the transfer bottling sector.

I) Label the tubes beforehand, if necessary.

J) Regulate and bottle the product following the quantities described in the Production Order, according to the variation limit specified.

K) Check the volume every 15 minutes, recording the values obtained in the Weight/Volume chart.

L) Perform the final packaging.

### Example 2

### Safety Margin

In order to demonstrate the safety of the invented formulation, safety tests were carried out with laboratory animals and with the target species (cats and dogs). The following tests were carried out:

1. Acute oral toxicity test for mice;

2. Acute Skin toxicity test for mice;

3. Dermal sensitisation test;

4. Safety test in dogs and cats.

The oral toxicity test in mice was carried out in order to collect information about the potential of oral lethality of the formulation in mice (Rattus norvegicus, Wistar line). The test used 6 animals (3 males and 3 females) that received the product orally in the dose of 2,000 mg/kg. The animals were observed for a period of 14 days for alterations in the skin, hair, eyes and mucous membranes, as well as dyspnoea, behavioural changes, shivering, convulsions, salivation, diarrhoea, lethargy, drowsiness, comatose and death. During the test period, no evident signs of toxicity were observed after the formulation was administered orally in the maximum recommended dose. Therefore, according to the GHS classification (Table 1 below), the toxicity of the product can be classified in category 5, and DL 50 of the product may be considered superior to the maximum recommended dose of 2,000 mg/kg.

TABLE 1
Toxicological classification, according to the GHS

| Category | DL50 | |
|---|---|---|
| Category 1 | 0-5 | mg/kg |
| Category 2 | >5-50 | mg/kg |
| Category 3 | >50-300 | mg/kg |
| Category 4 | >300-2,000 | mg/kg |
| Category 5 | >2,000 | mg/kg |

The skin toxicity test for mice was carried out in order to collect information about the potential of dermal toxicity of the formulation in mice (Rattus norvegicus, Wistar line). The test used 10 animals (5 males and 5 females), which had the back hair removed and shaved. These animals were weighed and identified individually with coloured pens. The volume used was calculated according to the body weight in the dose of 4,000 mg/kg and uniformly applied to an area of approximately 10% of the total area of the animal's body surface. In order to maintain the product in contact with the skin of the animal and preventingestion or inhaling, the animals were placed individually in small boxes, so as to hinder any movement. At the end of a period of 24 hours of exposure, the product residues were removed. The mice were observed for a period of 14 days for alterations in the skin, hair, eyes, mucous membranes, dyspnoea, behavioural changes, shivering, convulsions, salivation, diarrhoea, lethargy, drowsiness, coma and death. All animals presented an increase in weight during the test period. No other alterations were observed in the treated animals. According to the international protocol used, the acute skin toxicity may be considered superior to 4,000 mg/kg.

The dermal sensitisation test was carried out to collect information on the sensitising effects of the formulation on animal skin, defined by immunological reactions that are characterised by the appearance of oedemas and erythemas in laboratory animals. The test used 30 animals, which were divided into 2 groups (Control: 10 animals, and Treated: 20 animals). The treated group received 3 topical applications of the product without dilution, while the control group received 3 applications of deionized water, all at the same spot, for 2 consecutive weeks (day 0, day 6-8, day 13-15) for a period of 6 hours. The animals remained without treatment after the induction period was over, so as to allow the development of a hypersensitivity condition. On days 27-29 the challenge exposure was carried out. A patch soaked with the product was applied to the previously trichotomised right side (not treated) of all animals, and kept in that position for 6 hours. After 24 and 48 hours from removal of the patch, evaluations of the presence of erythema and oedema were made. The animals were weighted in the beginning and at the end of the test. In the test conditions, the formulation was classified as non-sensitising.

The safety test in cats and dogs was carried out in order to identify any dermatological or systemic reactions after application of the product. The test used 60 dogs and 48 cats of several breeds and ages, and both genders, divided into 5 groups of 12 animals for dogs, and 4 groups of 12 animals for cats:
12 dogs (6 males and 6 females) of several breeds and ages between 1 and 2 months (neonates) treated with the formulation;
12 dogs (6 males and 6 females) of several breeds and ages between 2 months and 1½ year (puppies) treated with the formulation;
12 dogs (6 males and 6 females) of several breeds and ages between 1½ year and 7 years (adults) treated with the formulation;
12 dogs (6 males and 6 females) of several breeds and ages beyond 7 years (elderly) treated with the formulation;
12 dogs, females, pregnant, in several gestation stages, treated with the formulation.
12 cats (6 males and 6 females) of several breeds and ages between 1 and 2 months (neonates) treated with the formulation;
12 cats (6 males and 6 females) of several breeds and ages between 2 months and 1½ year (kittens) treated with the formulation;
12 cats (6 males and 6 females) of several breeds and ages between 1½ year and 7 years (adults) treated with the formulation;
12 cats (6 males and 6 females) of several breeds and ages beyond 7 years (elderly) treated with the formulation;

The formulation showed to be safe when used in dogs of several breeds and ages, both genders, and in several gestation phases, not demonstrating any morphological or behavioural changes or signs of poisoning. The formulation did not cause foetal alterations or abortion in pregnant females.

### Example 3

### Pulicide Efficacy Tests of the Formulation in Dogs

The fipronil-based formulation plus an organic solvent, which acts as a transdermal carrier in this invention, in the spot-on form, demonstrated excellent pulicide activity (Ctenocephalides felis felis) in the controlled test with dogs.

The pulicide efficacy test used 12 dogs divided into 2 groups of 6 animals each:
Control Group: 6 dogs artificially infested with fleas and not treated;
Treated Group: 6 dogs artificially infested with fleas and treated with the formulation.

Each animal was infested with 100 unfed adult fleas (50 males and 50 females) from the laboratory colony. The animals were infested on days: −1, +5, +12, +19, +26, +33 and evaluated 48 hours after each infestation: days +2, +7, +14, +21, +28 and +35. The evaluations of the animals were carried out with the assistance of an appropriate fine-tooth comb to remove ticks. The recovered fleas were counted and fixed in a 70% alcohol solution.

The pulicide efficacy was calculated using the following formula: Efficacy percentage=(average number of living fleas recovered from the control group−average number of living fleas recovered from the treated group)/(average number of living fleas recovered from the control group)×100.

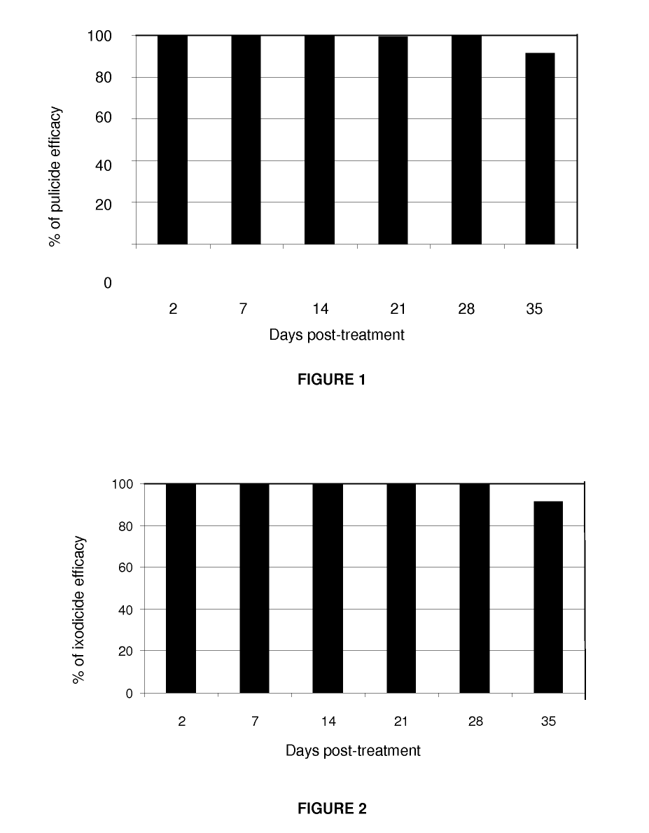

The formulation was effective in the control of fleas in dogs for up to 35 days, without the need of additional application, as shown in FIG. 1, where the bars in the chart indicate the percentage of pulicide efficacy of the spot on formulation in dogs during the days after the treatment. The continuous use of the formulation may lead to an environmental decontamination, extending the treatment period to up to 90 days.

### Example 4

### Pulicide Efficacy Tests of the Formulation in Cats

The fipronil-based formulation of this invention, in the spot-on form, demonstrated excellent pulicide activity (Ctenocephalides felis felis) in the controlled test with cats.

The pulicide efficacy test used 12 cats divided into 2 groups of 6 animals each:
Control Group: 6 cats artificially infested with fleas and not treated;
Treated Group: 6 cats artificially infested with fleas and treated with the formulation, according to the medical indications.

Each animal was infested with 100 unfed adult fleas (50 males and 50 females) from the laboratory colony. The animals were infested on days: −1, +5, +12, +19, +26, +33 and evaluated 48 hours after each infestation: days +2, +7, +14, +21, +28 and +35. The evaluations of the animals were carried out with the assistance of a fine-tooth comb, suitable for removing fleas. The recovered fleas were counted and fixed in a 70% alcohol solution.

The pulicide efficacy was calculated using the following formula: Efficacy percentage=(average number of living fleas recovered from the control group−average number of living fleas recovered from the treated group)/(average number of living fleas recovered from the control group)×100.

The formulation was effective in the control of fleas in cats for up to 35 days, without the need of additional application, as shown in FIG. 2, where the bars in the chart indicate the percentage of pulicide efficacy of the spot-on formulation in cats during the days after the treatment. The continuous use of the formulation may lead to environmental decontamination, extending the treatment period to up to 90 days.

### Example 5

### Ixodicide Efficacy Test in Dogs

The formulation of this invention, in the spot-on form, demonstrated excellent Ixodicide activity in the controlled test with dogs, regarding the Riphicephalus sanguineus.

The test used 12 animals divided into 2 groups of 6 animals each:
Control Group: 6 dogs artificially infested with ticks and not treated;
Treated Group: 6 dogs artificially infested with ticks and treated with the formulation, according to the leaflet indications.

Each animal was infested with 50 unfed adult ticks (25 males and 25 females) from the laboratory colony. The animals were infested on days: −2, +5, +12, +19, +26, +33 and evaluated 48 hours after each infestation: days +2, +7, +14, +21, +28 and +35. The evaluations of the animals were carried out with the assistance of a fine-tooth comb suitable for removing ticks. The recovered ticks were counted and fixed in a 70% alcohol solution.

The ixodicide efficacy was calculated using the following formula: Efficacy percentage=(average number of living ticks recovered from the control group−average number of living ticks recovered from the treated group)/(average number of living ticks recovered from the control group)×100.

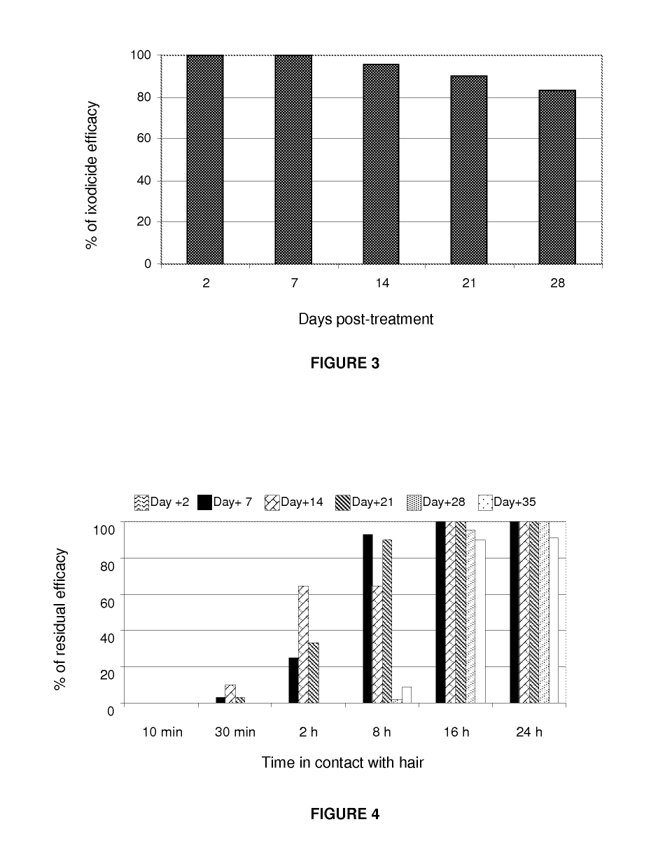

The formulation was effective in the control of ticks in dogs for up to 30 days, as shown in FIG. 3, where the bars in the chart indicate the percentage of ixodicide efficacy of the spot-on formulation in dogs along the days after the treatment.

### Example 6

### Evaluation of the Fipronil Residues on the Hair of Dogs Treated with the Formulation, in the Control of Evolutional Forms of Ctenocephalides felis felis Present in the Environment

In order to evaluate the residual effect on dog hair coat of treated with the formulation, 12 animals were tested:
Control Group: 6 animals were kept as control animals without treatment;
Treated Group: 6 animals were treated with the formulation.

Forty-eight hours after the treatment, the animals were submitted to trichotomy in different parts of the body (withers, back, base of the tail, belly, right and left sides). The trichotomised hair from each region was homogenised and placed in disposable Petri dishes duly identified with the day of the challenge, name of the animal, and group to which it belonged. The test used 0.02 g of hair of the corresponding group in each test tube. The challenges took place on days +7, +14, +21, +28e+35. All material related to the fleas are from the laboratory colony.

In order to evaluate the adulticide activity, 10 unfed adult fleas were used per test tube in six repetitions (one per dog), totalling 60 adults per group (control and treated groups). The test tubes were sealed with nylon and rubber band to prevent the subjects from escaping. On day +2, the hair of the dogs treated with the corresponding formulation and the hair of the dogs from the control group were added to the test tubes containing the duly identified adults. The material was evaluated after 10 minutes, 30 minutes, 2 hours, 8 hours, 16 hours, and 24 hours, with the aid of a stereoscopic microscope. The evaluation criterion was mobility, that is to say that fleas that could move were considered as being alive. New challenges were made using the same methodology described above on days +7, +14, +21, +28 and +35. The formulation was effective in assisting the control of adult fleas in the environment for up to 35 days after treatment (FIG. 4, where the chart represents the percentage of residual efficacy on hair of the dogs treated with the spot-on formulation in the control of adult fleas). The maximum effect thereof, above 90%, occurred after 16 hours of contact between the fleas and the treated animal's hair.

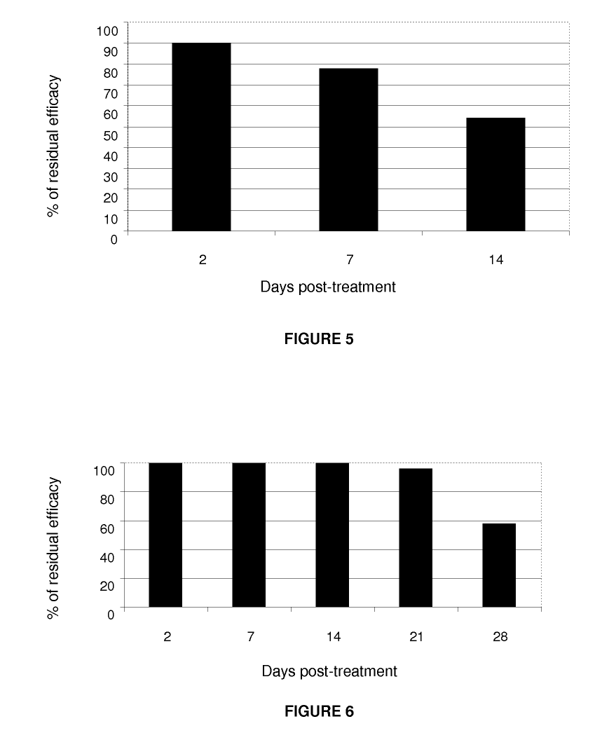

To evaluate the larvicide activity, 10 larvae of C. felis felis were used per test tube in six repetitions, totalling 60 larvae per group (control and treated group). The larvae were added along with 0.5 g of a diet required to maintain the larvae alive. The test tubes were sealed with nylon and rubber band. Twenty days after each challenge, the material was fixed in a 70% alcohol solution and was evaluated using a stereoscopic microscope in order to check if the flea cycle was successfully completed until the adult phase. The product was effective in assisting the control of larval forms in the environment for up to 14 days after the treatment (FIG. 5, where the chart represents the percentage of residual efficacy on the hair of the dogs treated with the formulation in controlling larvae).

To evaluate the ovicide activity, 10 eggs of C. felis felis were used per test tube in six repetitions, totalling 60 eggs per group (control and treated group). The test tubes were sealed with nylon and rubber band. After a period of 72 hours for each challenge, the material was fixed in a 70% alcohol solution and evaluated using a stereoscopic microscope. The product was effective in assisting the control of larval forms in the environment for up to 28 days after the treatment, as shown in FIG. 6, the chart of which represents the residual efficacy on the hair of the dogs treated with the formulation in controlling eggs.

### Example 7

### Post-Bath Efficacy Test

The ixodicide efficacy test in dogs after bathing used 24 dogs divided into 4 groups of 6 animals each:
Control Group: 6 animals, not bathed and not treated, were infested with 50 adult ticks (25 females and 25 males).

Group I: 6 animals, not bathed but treated with the formulation, were infested with 50 adult ticks (25 females and 25 males).
Group II: 6 animals treated with the formulation were infested with 50 adult ticks (25 females and 25 males) after having being bathed with a neutral soap only once.
Group III: 6 animals infested with 50 adult ticks (25 females and 25 males), treated with the formulation after having being bathed with a neutral soap. The dogs were bathed weekly.

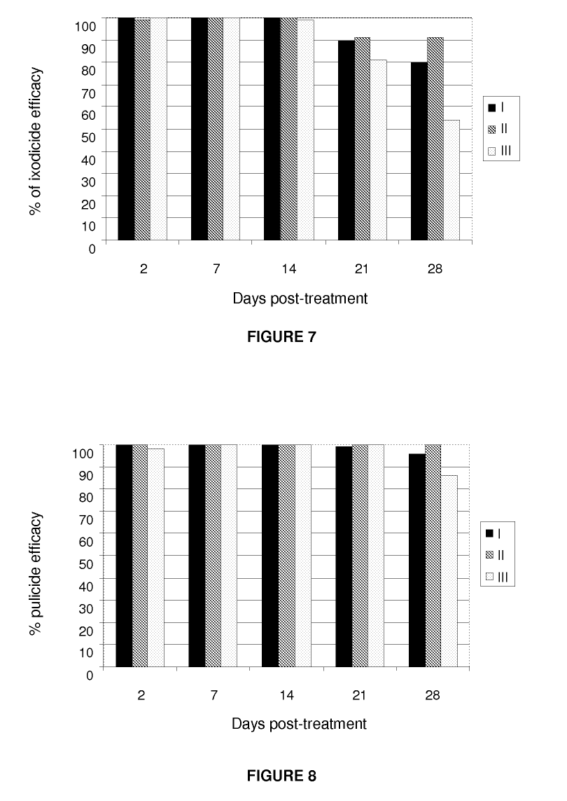

The results are depicted in FIG. 7, where the chart represents the ixodicide efficacy (%) after different post-bath periods in groups I (application without bath), II (application+single bath), and III (application+weekly bath).

The pulicide efficacy test in dogs after bath used 24 dogs divided into 4 groups of 6 animals each:
Control Group: 6 animals infested with 100 adult fleas (50 females and 50 males), not bathed and not treated.
Group I: 6 animals infested with 100 adult fleas (50 females and 50 males), not bathed but treated with the formulation.
Group II: 6 animals infested with 100 adult fleas (50 females and 50 males), treated with the formulation after having being bathed with a neutral soap only once.
Group III: 6 animals infested with 100 adult fleas (50 females and 50 males), treated with the formulation after having being bathed with a neutral soap. The dogs were bathed weekly.

The results are depicted in FIG. 8, where the chart represents the pulicide efficacy (%) after different post-bath periods in groups I (application without bath), II (application+single bath), and III (application+weekly bath).

### Example 8

### Miticide Efficacy Test

The miticide efficacy test was carried out for the treatment of itch mite (Sarcoptes scabei) infestation and ear mange (Otodectes cynotis). The test involving the fipronil-based formulation and the transdermal solvent carrier was carried out with naturally-infested dogs.

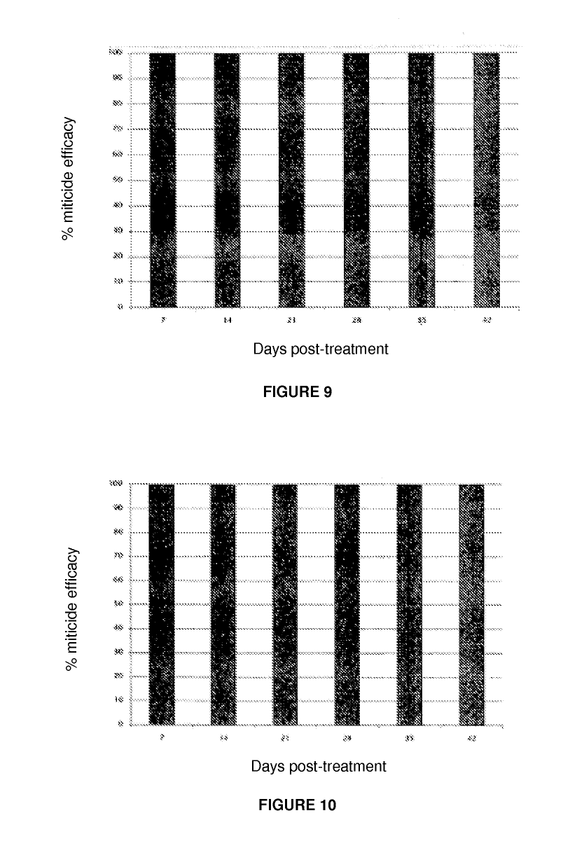

In the efficacy test for the itch mite infestation, the tested formula was applied topically on the back of the animal on day 0 (examination and treatment day of the animal) and day +15 (number of days after the first treatment). After that, all animals were evaluated until day +45 post-treatment, without any recurrence of the initial condition. The formulation was 100% effective in the treatment (FIG. 9, where the chart represents the miticide efficacy (%) for Sarcoptes scaeli after different post-application periods).

In the ear mange efficacy test, the tested formula was administered by applying 3 drops into the ears of the infested animals, with the remaining amount administered topically onto the back of the animals. The treatment was repeated on day +15 post-treatment, and the animals were evaluated until day +45, without any recurrence of the initial condition. The formulation was 100% effective in the treatment (FIG. 10, where the chart represents the miticide efficacy (%) for Otodectes cynotis after different post-application periods).

### Example 9

### Formulations

In order to obtain an ideal solution, this invention provides that, for each 100 mL, the formulation must contain the active ingredients and excipients according to the following qualitative-quantitative relationship:
FIPRONIL 10 g
BUTYLHYDROXYANIZOL 18 mg
BUTYLHYDROXYTOLUENE 9 mg
DIMETHYLSULFOXIDE 80 mL
ISOPROPYL ALCOHOL 100 mL
q.s.p.

1. A topical composition for controlling ectoparasites in dogs and cats, comprising fipronil and/or its derivates and salts, a transdermal carrier, antioxidants and vehicle.

2. Topical composition for controlling ectoparasites in dogs and cats, according to claim 1, wherein the transdermal carriers may be selected between: dimethylsulfoxide, ethylic alcohol, lactic acid, aliphatic alcohol containing 1 to 5 carbons, organic acids, propylene glycol and its derivates, isoparaffins, alkyl benzilic esters, dialkyl esters, benzil-benzylic esters, aliphatic ketones, aliphatic hydrocarbons, ethylene glycol and its derivates, polyalcohol pyrrolidones and its derivates, ethyl oleate.

3. Topical composition for controlling ectoparasites in dogs and cats, according to claim 2, wherein the transdermal carrier is preferably dymethylsulfoxide.

4. Topical composition for controlling ectoparasites in dogs and cats, according to claim 1, wherein the antioxidants are selected between: butylhydroxyanisol, butylhydroxytoluene, ascorbic acid, ascorbil palmitate, monothyoglycerol, propylgallate, sulphur dioxide, tocopherol, tocopherol acetate, oil solutions of tocopherol.

5. Topical composition for controlling ectoparasites in dogs and cats, according to claim 4, wherein the antioxidants are preferably butylhydroxyanisol and butylhydroxytoluene.

6. Topical composition for controlling ectoparasites in dogs and cats, according to claim 1, wherein the vehicle is selected between isopropylic alcohol, ethylic alcohol, propylene glycol.

7. Topical composition for controlling ectoparasites in dogs and cats, according to claim 6, wherein the vehicle is preferably isopropylic alcohol.

8. Topical composition for controlling ectoparasites in dogs and cats, according to claim 1, wherein the concentration of the active ingredient varies between 50 mg/mL and 200 mg/mL.

9. Topical composition for controlling ectoparasites in dogs and cats, according to claim 8, employing 100 mg/mL of fipronil or the equivalent concentration of its derivates or salts.

10. Topical composition for controlling ectoparasites in dogs and cats, according to claim 5, wherein the concentration of antioxidants varies between 0.144 and 0.216 mg/mL of butylhydroxyanisol, and between 0.072 and 0.108 mg/mL of butylhydroxytoluene.

11. Topical composition for controlling ectoparasites in dogs and cats, according to claim 10, comprising 0.18 mg/mL of butylhydroxyanisol, and 0.09 mg/mL of butylhydroxytoluene.

12. Topical composition for controlling ectoparasites in dogs and cats, according to claim 3, wherein the concentration of dimethylsulfoxide is between 50% and 90% of the formulation.

13. Topical composition for controlling ectoparasites in dogs and cats, according to claim 12, wherein the concentration of dymethylsulfoxide is preferably 80%.

14. Topical composition for controlling ectoparasites in dogs and cats, according to claim 1, comprising:
Fipronil;
Dimethylsulfoxide;
Butylhydroxyanisol;
Butylhydroxytoluene;
Isopropylic alcohol.

15. Topical composition for controlling ectoparasites in dogs and cats, according to claim 1, characterized by being employed in the combat against fleas, acari, and ticks.
